(19) United States
(12) Plant Patent Application Publication
(10) Pub. No.: US 2003/0084489 P1
(43) Pub. Date: May 1, 2003

(54) Miniature rose plant named 'Meirosfon'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/307,962

(22) Filed: Dec. 3, 2002

Related U.S. Application Data
(63) Continuation of application No. 09/597,230, filed on Jun. 20, 2000, now abandoned.

(51) Int. Cl. A01H 5/00

(52) U.S. Cl. PLT/122

(57) ABSTRACT

A new and distinct variety of rose plant of the Miniature Class is provided which abundantly forms clusters of attractive double red blossoms having a light base. Commonly the blossoms only partially open even at maturity. The growth habit is bushy. Decorative medium green semi-glossy foliage is formed that contrasts nicely with the red blossoms. The plant can be readily propagated from cuttings and is particularly well suited for growing as an attractive potted gift plant.

### SUMMARY OF THE INVENTION

The new variety of Miniature rose plant was created by artificial pollination wherein two parents were crossed which previously had been studied in the hope that they would contribute the desired characteristics. The female parent (i.e., the seed parent) of the new variety was the product of the cross of the ‘Meijikatar’ variety (U.S. Plant Pat. No. 4,682) and the ‘Meilipo’ variety (U.S. Plant Pat. No. 8,504). The male parent (i.e., the pollen parent) was the ‘Meivilou’ variety (non-patented in the United States). The parentage of the new variety can be summarized as follows:

(‘Meijikatar’×‘Meilipo’)×‘Meivilou’.

The seeds resulting from the above pollination were sown and small plants were obtained which were physically and biologically different from each other. Selective study resulted in the identification of a single plant of the new variety.

It was found that the new variety of Miniature rose plant of the present invention possesses the following combination of characteristics:

(a) exhibits a bushy growth habit,

(b) forms in abundance clusters of attractive double red blossoms having a light base that commonly only partially open,

(c) forms decorative medium green semi-glossy foliage,

(d) can be readily propagated from cuttings, and

(e) is particularly well suited for growing as an attractive potted gift plant.

The profusion of red blossoms contrast nicely with the medium green foliage.

The new variety well meets the needs of the horticultural industry. It is particularly well-suited for pot forcing under greenhouse growing conditions to form an attractive gift plant.

The new variety has been found to undergo asexual propagation in France by a number of routes, including budding, grafting, and cuttage. Asexual propagation by the above-mentioned methods as performed in the South of France has shown that the characteristics of the new variety are strictly transmissible from one generation to another.

The new variety has been named the ‘Meirosfon’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were six months of age and were observed during September while growing on their own roots in greenhouses at LeCannet des Maures, Var, France. Dimensions in centimeters are shown at the bottom of the photograph.

FIG. 1—illustrates a specimen of a young shoot;

FIG. 2—illustrates specimens of two floral buds before the opening of the sepals;

FIG. 3—illustrates specimens of two floral buds at the opening of the sepals;

FIG. 4—illustrates specimens of two floral buds at the opening of the petals;

FIG. 5—illustrates specimens of two flowers during the course of opening;

FIG. 6—illustrates specimens of two open flowers—plan view—obverse;

FIG. 7—illustrates specimens of two open flowers—plan view—reverse;

FIG. 8—illustrates specimens of two fully open flowers—plan view—obverse;

FIG. 9—illustrates specimens of two fully open flowers—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates specimens of two leaves with three leaflets with the upper surface being shown at the upper left and the under surface being shown at the lower right;

FIG. 15—illustrates specimens of two leaves with five leaflets with the upper surface being shown at the lower left and the under surface being shown at the upper right;

and

FIG. 16—illustrates specimens of two leaves with seven leaflets with upper surface being shown at the bottom and the under surface at the top.

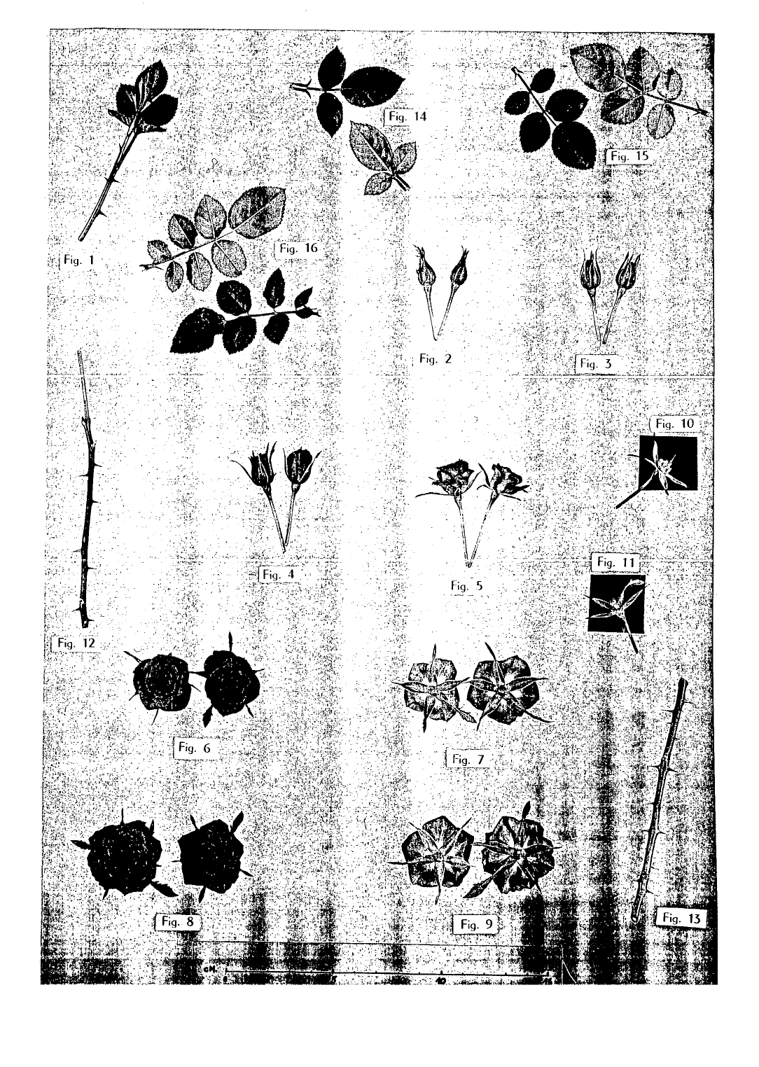

### DETAILED DESCRIPTION

The chart used in the identification of the colors is that of The Royal Horticultural Society (R.H.S. Colour Chart). The terminology which precedes reference to the chart has been added in some instances to indicate the corresponding colors in more common terms. The description is based on the observation of six month old specimens of the new variety which were observed during April while growing on their own roots in greenhouses at LeCannet des Maures, Var, France.

Class: Dwarf Miniature.

Plant:

Height.—approximately 30 to 35 cm on average at the end of the growing season.

Habit.—bushy.

Branches:

Color.—young stems: medium green, Green Group 143A. adult wood: dark green, near Green Group 137B. thorns.—size: medium. quantity: moderately sparse (as illustrated). color: near Yellow-Green Group 153C on young stems. configuration: fairly straight and elongated.

Leaves:

Petioles.—upper surface: non-glandular, and near Green Group 137A in coloration. under surface: lighter green with a few prickles.

Stipules.—adnate, pectinate, and rather broad.

Leaflets.—number: 3, 5, and 7 (most often). shape: with a symmetrical tip and a rounded base (as illustrated). texture: strong. serration: regular. general appearance: very dense, medium green and semi-glossy. color (young foliage): upper surface: near Green Group 137A. under surface: near Yellow-Green Group 147B. color (adult foliage): upper surface: near Green Group 137A. under surface: near Yellow-Green Group 147B.

Inflorescence:

Number of flowers.—commonly approximately 2 to 5 per stem.

Peduncle.—rigid, near Yellow-Green 144A, and the length commonly is approximately 2.5 to 3 cm on average.

Sepals.—upper surface: tomentose, and near Green Group 143B. under surface: glabrous and near Yellow-Green Group 144A. size: elongated, commonly approximately 2 to 3 cm in length, and with some short extensions (as illustrated).

Buds.—shape: conical. size: medium for class, and approximately 1.5 cm in length.

Flower.—form: double and cup-shaped. diameter: approximately 3 cm on average when fully open. color (when opening begins): upper surface: near Red-Purple Group 58B suffused with Red-Purple Group 57A. under surface: near Red-Purple Group 57C with lightening at the base. color (when blooming): upper surface: near Red-Purple Group 57C suffused with Red-Purple Group 57A. under surface: near Red-Purple Group 57C with lightening at the base. color (at end of opening): upper surface: near Red-Purple Group 57C suffused with Red-Purple Group 57A. under surface: near Red-Purple Group 57B with lightening at the base. fragrance: none. anthers: near Yellow-Orange Group 21A. stamen number: approximately 40 to 45 on average. filaments: yellow, near Green-Yellow Group 1B. pistil number: approximately 20 to 25 on average. petal configuration: wedge-shaped base and a reflexed tip. stigmas: near Yellow Group 8C. styles: near Green-Yellow Group 1D. receptacle: near Yellow-Green Group 144A. lasting quality: very long on the plant. petal drop: good, the petals detach well.

Development:

Vegetation.—strong.

Blooming.—very abundant.

Resistance to diseases.—very good, especially with respect to Powdery Mildew.

Aptitude to bear fruit.—good, forms hips that are pear-shaped in longitudinal section.

I claim:

1. A new and distinct variety of Miniature rose plant characterized by the following combination of characteristics:
(a) exhibits a bushy growth habit,
(b) forms in abundance clusters of attractive double red blossoms having a light base that commonly only partially open,
(c) forms decorative medium green semi-glossy foliage,
(d) can be readily propagated from cuttings, and
(e) is particularly well suited for growing as an attractive potted gift plant;
substantially as herein shown and described.
